(19) United States
(12) Patent Application Publication
(10) Pub. No.: US 2024/0226256 A1
(43) Pub. Date: Jul. 11, 2024

(54) Combination Therapy Tumour Cell Vaccine

(71) Applicant: Queen's University at Kingston, Kingston (CA)

(72) Inventors: Kyle Seaver, Woodbridge (CA); Sam Basta, Kingston (CA); Katrina Gee, Joyceville (CA)

(21) Appl. No.: 18/561,772

(22) PCT Filed: May 19, 2022
(86) PCT No.: PCT/CA2022/050792

§ 371 Date: Nov. 17, 2023

Related U.S. Application Data
(60) Provisional application No. 63/190,504, filed on May 19, 2021.

(51) Int. Cl. A61K 39/00; A61P 35/00; A61P 37/06

(52) U.S. Cl. CPC A61K 39/0011; A61P 35/00; A61P 37/06; A61K 2039/5152; A61K 2039/55527; A61K 2039/55561

(57) ABSTRACT

A cancer vaccine includes at least one tumour associated antigen (TAA), at least one Toll-like receptor (TLR) agonist, at least one cytokine, and a pharmaceutically acceptable vehicle. The at least one TAA may be provided by dead tumour cells, such as γ-irradiated tumour cells or lysis and UV treated tumour cells, the at least one TLR agonist may comprise 5 CpG-1826 and the at least one cytokine may comprise IL-27. When administered to a mammalian subject the cancer vaccine prevents, inhibits, or slows tumour development in the subject, and the vaccine may provide a long-term T cell activation and memory against tumour development in the subject.

### FIELD

This invention relates generally to vaccines for treating cancer. More specifically, the invention relates to cancer vaccines based on a combination of dead tumour cells and one or more adjuvants.

### BACKGROUND

Cancer vaccines are an immunotherapy that can be used prophylactically to prevent tumour establishment, or therapeutically to reduce disease progression. The implementation of cancer vaccines has expanded beyond viral-associated cancers, such as those caused by HBV and HPV [1]. Many cancer vaccines aim to promote T cell mediated killing of cancer cells by enhancing recognition of tumour associated antigen (TAA) [1]. TAAs represent a target for the immune system to identify cancer cells and develop a cytotoxic immune response against them. With new technology advancing the field of cancer diagnostics and biomarker discovery, novel TAAs called neoantigens are being discovered, improving cancer vaccine development [2,3]. Despite discovering new TAAs, selecting the desired TAA that can mount a potent and specific anti-tumour immune response needs to be addressed. Providing access to a wider range of antigens can avoid the need to select one specific TAA [4]. The discovery of these neoantigens can also identify patients at high risk of developing a particular type of cancer allowing for effective implementation of prophylactic cancer vaccines in the clinic. Improving access to a wide range of TAAs can be accomplished using whole tumour cells that have been exposed to a method of inducing cell death such as exposure to irradiation or consecutive cycles of freeze/thaw (F/T) [5]. However, whole tumour cells alone are not able to provide a potent anti-tumour immune response capable of providing long-term protection. Therefore, adjuvants are added to enhance the activation of a desired immune response.

Adjuvants such as pathogen associated molecular patterns (PAMPs), cytokines, chemokines, and other small molecules have been used in whole tumour cell vaccines [6-9]. PAMPs are recognized by pattern recognition receptors, of which Toll-like receptors are the most well studied and understood. Among Toll-like receptors, TLR1, -2, -4, -5, -6, and -10 are localized to the cell surface while TLR3, -7, -8, and -9 are localized to the endosomal membrane. TLRs have been studied for their potential use in cancer therapy [10,11]. The TLR9 agonists CpG-ODNs have demonstrated promise as cancer vaccine adjuvants in prophylactic and therapeutic models [12]. Cytokines such as interleukin-12 (IL-12) and GM-CSF have also been used as cancer vaccine adjuvants [13,14], and they can be used independently or in combination with each other to help further promote an anti-tumour immune response [15,16]. However, despite the success of IL-12 in promoting anti-tumour immunity, it has failed to be translated into the clinic as a result of toxicity concerns [17]. The cytokine IL-12 is a member of the cytokine family that includes IL-27, IL-30, and IL-35, each having a unique contribution to tumour development [18]. Although IL-27 has been presented as a pleiotropic cytokine, use in cancer immunotherapy has largely focused on constitutive expression of IL-27 by transfecting cancer cells, and previous work has focused on delivering IL-27 by the laborious process of stably transfecting cancer cells, or via a viral vector such as adenovirus to ensure adjuvant and antigen are present simultaneously.

### SUMMARY

According to one aspect of the invention there is provided a composition, comprising: at least one tumour associated antigen (TAA); at least one TLR agonist; at least one cytokine; and a pharmaceutically acceptable vehicle. The composition may be a cancer vaccine. The cancer vaccine may prevent, inhibit, or slow melanoma development in a subject. The composition may provide long-term T cell activation and memory against B16 tumour development in a subject.

According to another aspect of the invention there is provided a method for preventing, inhibiting, or slowing tumour development, comprising: providing a composition comprising at least one tumour associated antigen, at least one TLR agonist, at least one cytokine, and a pharmaceutically acceptable vehicle; administering an effective amount of the composition to a subject; wherein the composition prevents, inhibits, or slows the tumour development in the subject. The method may provide long-term T cell activation and memory against B16 tumour development in the subject.

In some embodiments, the at least one TAA comprises dead tumour cells.

In some embodiments, the dead tumour cells are not necrotic.

In some embodiments, the at least one TLR agonist comprises a TLR9 agonist.

In one embodiment, the at least one TLR9 agonist comprises a CpG oligodeoxynucleotide (CpG-ODN).

In one embodiment, the TLR9 agonist comprises CpG-1826.

In some embodiments, the at least one cytokine comprises an interleukin (IL).

In one embodiment, the cytokine comprises IL-27.

In one embodiment, the TLR9 agonist comprises CpG-1826 and the cytokine comprises IL-27.

In one embodiment, the dead tumour cells comprise γ-irradiated tumour cells.

In one embodiment, the dead tumour cells comprise lysis and UV treated tumour cells.

In one embodiment, the dead tumour cells comprise γ-irradiated or lysis and UV treated B16 tumour cells.

In one embodiment, the dead tumour cells comprise γ-irradiated or lysis and UV treated B16 tumour cells, and the cancer vaccine prevents, inhibits, or slows melanoma development.

### BRIEF DESCRIPTION OF THE DRAWINGS

For a greater understanding of the invention, and to show more clearly how it may be carried into effect, embodiments will be described, by way of example, with reference to the accompanying drawings, wherein:

FIG. 1 is a schematic representation of a vaccination schedule used for the development of a prophylactic cancer vaccine, according to one embodiment.

FIGS. 2A-2C are plots of tumour growth in mice after injection with a prophylactic cancer vaccine as indicated and challenged with live B16-OVA tumour cells on day 0, wherein tumour outgrowth was measured every second day (N=5).

FIG. 2D is a Kaplan-Meyer survival analysis of the vaccinated mice in FIGS. 2A-2C following live tumour engraftment.

FIG. 2E is a Kaplan-Meier survival curve comparing vaccination schedules of either one or two dead cancer cell vaccine deliveries, wherein mice were challenged with live tumour cells one week following final vaccination (N=3 for each treatment).

FIG. 3A is a plot of tumour volume in mice following injection with dead B16-OVA cancer cells exposed to different methods of inducing cell death (UV exposed dead cancer cells (“UV”), F/T dead tumour cells (“Lysis”) or combination of lysis and UV (“LyUV”) (N=3 for each treatment).

FIG. 3B is a Kaplan-Meier survival curve for the mice in FIG. 3A.

FIG. 3C is a Kaplan-Meier survival curve for mice following injection with dead B16-OVA cancer cells exposed to different methods of inducing cell death (UV exposed dead cancer cells (“UV”), F/T dead tumour cells (“Lysis”), combination of lysis and UV (“LyUV”), or γ-irradiation (N=at least 6 for each treatment).

FIGS. 4A and 4B are Kaplan-Myer survival curves of mice (N=3 to 7, depending on treatment) vaccinated with PBS, or with γ-irradiated tumour cells either alone or with IL-27 (10 ng/mouse or 100 ng/mouse), following challenge at day 0 with live B16-OVA cells according to the schedule of FIG. 1.

FIG. 4C is a Kaplan-Myer survival curve of mice treated with LyUV cells together with IL-27 (10 ng/mouse or 100 ng/mouse), following challenge at day 0 with live B16-OVA cells according to the schedule of FIG. 1.

FIG. 5A is a Kaplan-Myer survival curve of mice (N=3 to 6, depending on treatment) vaccinated with PBS or with γ-irradiated dead tumour cells and CpG (2 μg/mouse or 20 μg/mouse), following challenge at day 0 with live B16-OVA cells according to the schedule of FIG. 1.

FIG. 5B is a Kaplan-Meyer survival curve for PBS vaccinated control mice and mice that were vaccinated with γ-irradiated tumour cells or γ-irradiated tumour cells and CpG (20 μg/mouse) (N=6).

FIG. 6 is a Kaplan-Meyer survival curve of mice vaccinated with γ-irradiated tumour cells in combination with IL-27 (10 ng/mouse) and CpG-1826 (20 μg/mouse), N=3 for each treatment.

FIG. 7 is a Kaplan-Meyer survival curve for mice that received PBS (control) or a vaccine of dead tumour cells (exposed to F/T and UV (“LyUV”) alone or with CpG (20 μg/mouse), IL-27 (10 ng/mouse), or CpG (20 μg/mouse) and IL-27 (10 ng/mouse), N=3 for each treatment.

FIG. 8 is a schematic representation of a prophylactic cancer vaccine delivery schedule according to one embodiment used for mice, with live tumour engraftment on day 0, in which tumour free mice were rechallenged at day 60, and tumour outgrowth was determined.

FIG. 9 is a plot showing tumour growth in mice that were vaccinated with γ-irradiated tumour cells and either IL-27 (10 ng/mouse), CpG (20 μg/mouse), or IL-27 (10 ng/mouse) and CpG (20 μg/mouse) and had no palpable tumours after 60 days and then rechallenged with live tumour cells according to the schedule in FIG. 8; tumour growth was measured every second day (N=3).

FIGS. 10A and 10B are survival plots for mice that received PBS (control) or a vaccine of dead tumour cells (exposed to γ-irradiation (FIG. 10A) or LyUV (FIG. 10B) alone or with IL-27 (10 ng/mouse) or IL-27 (100 ng/mouse), and were rechallenged with live tumour cells according to the schedule in FIG. 8; N=at least 6 for each treatment.

FIG. 11 is a survival plot for mice that received PBS (control) or a vaccine of dead tumour cells (exposed LyUV) alone or with IL-27 (10 ng/mouse) or with IL-27 (10 ng/mouse) and CpG (20 μg/mouse), and were rechallenged with live tumour cells according to the schedule in FIG. 8; N=at least 5 for each treatment.

FIGS. 12A-12E are plots showing effectiveness of other adjuvants in combination with IL-27 as measured by cell surface marker expression on day 7 bone marrow-derived dendritic cells.

FIG. 13A is a schematic diagram of a therapeutic vaccine delivery schedule, according to one embodiment.

FIGS. 13B and 13C are plots of tumour volume and survival for mice that received PBS (control) or a vaccine of dead tumour cells (exposed LyUV) alone or with IL-27 (10 ng/mouse), with CPG (20 ng/mouse), or with IL-27 (10 ng/mouse) and CpG (20 μg/mouse) according to the schedule of FIG. 13A; N=3 for each treatment.

### DETAILED DESCRIPTION OF EMBODIMENTS

According to a broad aspect of the invention, there are provided cancer vaccine compositions and methods for use thereof. The cancer vaccine compositions include a combination of active ingredients and a pharmaceutically acceptable vehicle. The combination of active ingredients may include at least one TLR agonist, at least one cytokine, and an antigen source such as dead tumour cells. As noted above, prior work has focused on the use of cytokines or TLR agonists individually as adjuvants in cancer vaccines. In contrast, embodiments described herein relate to cancer vaccines comprising combinations of cytokines and TLR agonists as cancer vaccine adjuvants together with dead tumour cells as an antigen source. The pharmaceutical compositions may be administered to subjects, e.g., mammals, and particularly humans, prophylactically for the prevention of cancer, and therapeutically for the treatment of cancer. Treatment may include inhibiting, slowing, or eradicating tumour development in subjects.

As used herein, the term “dead tumour cells” refers to tumour cells that have been subjected to a treatment that renders them non-viable (e.g., incapable of replicating) and preserves tumour cell antigenic features that contribute to efficacy of a vaccine as described herein. Such tumour cell antigenic features may include, for example, surface characteristics such as one or more cell surface proteins and/or other proteins (i.e., TAAs) against which an immune response is induced in a subject having received a cancer vaccine according to an embodiment. For example, dead tumour cells used in embodiments may be prepared by subjecting tumour cells to one or more treatments such as, but not limited to, irradiation (e.g., γ-irradiation, ultra-violet (UV) irradiation, or irradiation using other wavelengths, such as visible light) or a treatment that induces apoptosis such as plasma irradiation, or lysis (e.g., freeze/thaw cycles). The dead tumour cells, which may also be referred to as antigen donor cells, may be suitably purified for inclusion in a cancer vaccine composition to be administered to a subject.

According to embodiments, the at least one TLR agonist may comprise a TLR9 agonist including CpG oligodeoxynucleotide (CpG-ODN). Examples of TLR9 agonists include but are not limited to any unmethylated cytosine-phosphate-guanine dinucleotides (ODNs) (including but not limited to 1585, 2216, 2336, 1826, 2006, 2007, M362, 2395), repeated ODNs (e.g., SD-101, IMO-2125, IMO-2055, ISS 1018), small molecules (e.g., hydroxychloroquine), double-stem loop immunomodulators (dSLIM) family of molecules (e.g., MGN1703), spherical nucleic acids (SNAs) (e.g., AST-008), encapsulated TLR9 agonist (e.g., NZ-TLR9), and virus-like particles (VLP) containing capsid proteins that encapsule TLR9-ODNs (e.g., CMP-001). In one embodiment, the at least one TLR9 agonist comprises CpG-1826. Additional TLR agonists may include, but are not limited to, those found in Kaczonowska et al [11]. A non-exhaustive list includes TLR1/TLR2 agonists (triacylated lipoproteins, lipoteichoic acid, peptidoglycans, Zymosan, Pam₃CSK₄,) TLR2/TLR6 (diacylated lipopeptides), TLR3 (Poly I:C, and dsDNA nucleic acids). TLR4 (LPS), TLR-5 (flagellin), TLR7/TLR8 (e.g., ssRNA or an imidazoquinoline such as R848), TLR9 (unmethylated CpG-DNA).

According to embodiments, the at least one cytokine comprises an interleukin (IL) which may include IL-2, IL-6, IL-12, IL-15 or IL-23. In one embodiment, the at least one cytokine comprises IL-27. In other embodiments, the at least one cytokine may comprise interferon-gamma, granulocyte-macrophage colony-stimulating factor (GM-CSF), interferon-alpha, or transforming growth factor beta (TGF-β).

Previous work has focused on delivering IL-27 by stably transfecting cancer cells, or via a viral vector such as adenovirus to ensure adjuvant and antigen are present simultaneously. These are laborious processes that may limit practical use. In contrast, embodiments described herein use recombinant IL-27 administered with dead tumour cells in the absence of viral vectors and transfected cancer cells. As described herein, using this approach antigen and adjuvant may be delivered to the same site of the patient, with the added advantage of controlling antigen and adjuvant concentration independently. Indeed, control of IL-27 concentration, considering its pleotropic effects in cancer, is a critical component to developing a successful cancer vaccine. Exemplary embodiments presented herein are the first use of a cancer vaccine that combines a cytokine, recombinant IL-27, with a TLR9 agonist and a source of dead tumour cells. Thus, the invention provides a novel adjuvant combination for a cancer vaccine without the need for transfection or use of viral vectors for cytokine delivery.

As used herein, the “administration” of a cancer vaccine composition to a subject includes any route or routes of introducing or delivering to a subject the composition to perform its intended function. Administration can be carried out by any suitable route, including orally, intranasally, intraocularly, parenterally (intravenously, intramuscularly, intrathecally, epidurally, intracranially, intraperitoneally, or subcutaneously), or topically, or by a combination thereof. Parenteral administration of the composition is generally characterized by intra-tumour injection. Injectables can be prepared in conventional forms, either as liquid solutions or suspensions, solid forms suitable for solution of suspension in liquid prior to injection, or as emulsions.

The exact amount of a composition required will vary from subject to subject, depending on the species, age, weight, and general condition of the subject, the stage and location of the cancer being treated, the mode of administration, and the like. Thus, it is not possible to specify an exact amount for every composition. However, an appropriate amount can be determined by one of ordinary skill in the art using only routine experimentation given the teachings herein.

As used herein, the term “effective amount” or “pharmaceutically effective amount” or “therapeutically effective amount” or “prophylactically effective amount” of a composition is a quantity sufficient to achieve or maintain a desired therapeutic and/or prophylactic effect, e.g., an amount which results in the prevention of, or a decrease in, the cancer being treated, e.g., of a tumour.

As described herein, the compositions may be administered in vivo in a pharmaceutically acceptable carrier. The term “pharmaceutically acceptable” refers to a substance that is not biologically or otherwise undesirable, i.e., the substance is physiologically compatible and may be administered to a subject without causing any undesirable biological effects or interacting in a deleterious manner with any of the other components of the pharmaceutical composition in which it is contained. The carrier would be selected to minimize any degradation of the active ingredient(s) and to minimize any adverse side effects in the subject, as would be well known to one of skill in the art.

As used herein, the term “pharmaceutically acceptable carrier” is intended to include any and all solvents, dispersion media, coatings, antibacterial and antifungal compounds, isotonic and absorption delaying compounds, and the like, compatible with pharmaceutical administration. Suitable carriers and their formulations are described in Remington: The Science and Practice of Pharmacy (19th ed.) ed. A. R. Gennaro, Mack Publishing Company, Easton, Pa. 1995. For example, an appropriate amount of a pharmaceutically acceptable salt may be used in the formulation to render the formulation isotonic. Examples of the pharmaceutically acceptable carriers include, but are not limited to, saline, Ringer's solution, and dextrose solution. The pH of the solution is preferably from about 5 to about 8, and more preferably from about 7 to about 7.5. Further carriers may include sustained release preparations such as semipermeable matrices of solid hydrophobic polymers containing the active ingredient(s), which matrices are in the form of shaped articles, e.g., films, liposomes, or microparticles. It will be apparent to those of ordinary skill in the art that certain carriers may be more preferable depending upon, for instance, the route of administration and concentration of composition being administered.

The invention will be further described by way of the below examples wherein it is demonstrated that embodiments comprising γ-irradiated dead tumour cells or lysed and UV-irradiated dead tumour cells in combination with a TLR agonist and a cytokine (e.g., CpG-ODN and recombinant IL-27) prevents, inhibits, or slows B16 melanoma development in a mouse model. It will be appreciated that whereas recombinant IL-27 was used in the example, other forms of IL-27 may also be used, such as that obtained from a transfected cell line. However, use of recombinant (exogenous) cytokine conveniently allows for control of cytokine concentration, whereas cytokine produced from transfected cell lines (endogenous) relies on cell number to control cytokine concentration, which may be less convenient. In view of toxicity concerns with certain cytokines with IL-12, exogenous IL-27 was used to provide greater control over the concentration of IL-27 administered.

Whereas the studies presented herein provide exemplary dosages of IL-27 and CpG, it will be appreciated that other dosages may be used. For example, for IL-27 it is expected that dosages of 5, 10, 15, 20, 25, or 50 ng/mouse would be effective, and for CpG it is expected that dosages of 2, 5, 7.5, 10, or 20 μg/mouse would also be effective. Further, an effective dosage range can be determined (e.g., scaled) according to factors such as body mass or metabolic rate as is known in the art, for use in other subjects such as humans. Indeed, studies are routinely conducted in mammalian models such as mice and the treatment regimes may be readily adapted and scaled to other mammals, including humans. The B16 melanoma cell line used in the example is a model of murine melanoma in which there are numerous antigenic similarities with humans for T cell recognition [19]. With T cells being actively involved in this model, it is expected that a combination therapy according to teachings and embodiments herein would translate effectively to human melanoma and other cancers. Furthermore, the development of human malignant melanoma resembles that of B16 melanoma, thus the latter is an effective model for studying vaccine development.

Specific features described in the below examples, such as dosage and administration schedule, may be varied, and the approach may be adapted for other types of cancers and other subjects, as would be apparent to those of ordinary skill in the art. Accordingly, the examples are not to be construed as limiting the invention in any way.

### Examples

### Methods

Mice, Cell Lines, and Reagents Male and Female C57BL/6 (H-2^{b}) mice (6-8 weeks old) were purchased from Jackson Laboratories and kept in pathogen-free conditions. All animal experiments were conducted in accordance with the Canadian Council of Animal Use and approved by Queen's University Animal Care Services. B16-OVA (H-2^{b}) cells (a gift from NIH) are an adherent melanoma cancer cell line that constitutively expresses chicken ovalbumin. The B16-OVA cancer cells were maintained in DMEM supplemented with 5% FCS and 500 μg/mL of G418. Cell lines were maintained in a 37° C. incubator with 5% humidified CO₂ atmosphere. Recombinant murine IL-27 was purchased from Biolegend. CpG oligodinucelotide-1826 was purchased from Invivogen.

### Preparation of Cancer Vaccine

For the development of a whole tumour cell vaccine B16-OVA cells were harvested and seeded into microcentrifuge tubes at a density of 5.0×10⁶ cells/200 μL in PBS. It is noted that other cell densities may also be used, for example, 1.0×10⁶ cells/200 μL, or 2.5×10⁶ cells/200 μL. The cells were then exposed to 60 Greys (Gys) of γ-irradiation (Cs¹³⁷ GammaCell as irradiator source) at a dose of 22.3 Gys/hour. Cancer cells subjected to lysis were exposed to repeated freeze/thaw (F/T) cycles resulting in the induction of necrosis. For F/T, the B16-OVA cells were frozen in liquid nitrogen and then immediately placed in a 37° C. water bath until thawed, this process was repeated for a total of five cycles. Cancer cells subjected to ultra-violet (UV) treatment were exposed to 10,000 mj/cm³ for 10 minutes using a CL-1000 UltraViolet Crosslinker. Cancer cells subject to LyUV treatment underwent a single round of F/T followed by immediate exposure to 10,000 mj/cm³ for 10 minutes.

In a first study, one cohort of mice (“lysis”, N=5) received a vaccine containing F/T dead tumour cells. A second cohort (“irradiation”, N=5) received a vaccine containing γ-irradiated dead tumour cells, and a third (control) cohort (“PBS”, N=5) received PBS. These were delivered via intraperitoneal injections on days −14 and −7. On day 0 all mice were challenged with 1.0×10⁶ live B16-OVA cells in 100 μL of PBS via subcutaneous injection in the right hind flank of each mouse. Tumour outgrowth was measured every second day using calipers and volume was determined according to the formula (l×w²) 2 (where w is the smaller of the two parameters). Mice were euthanized once tumour volume reached 2000 mm³.

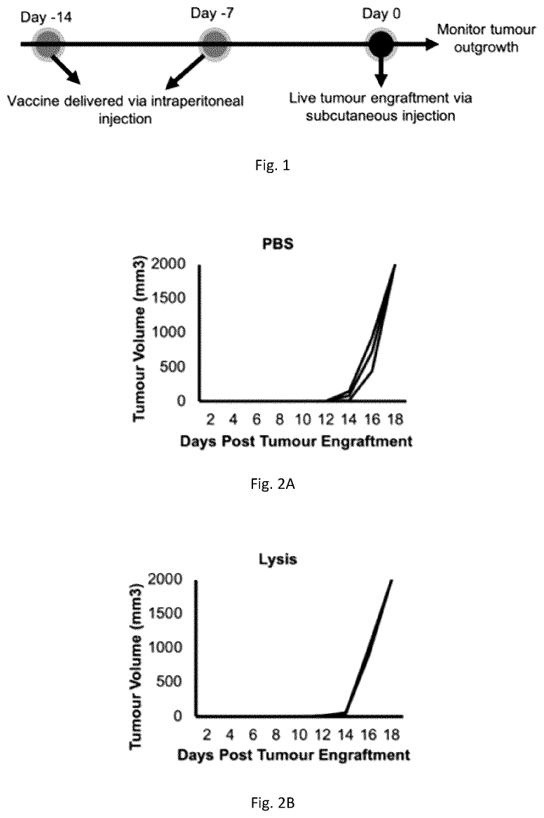

In a second study to determine if the vaccine schedule could be shortened by providing only a single delivery of dead tumour cells, cohorts of mice (N=3 per treatment) received a single injection of either F/T dead tumour cells “lysis (1 injection)”, γ-irradiated dead tumour cells “gamma-irr (1 injection)”, or PBS “PBS”. These were delivered via intraperitoneal injections on day −7. In addition, cohorts of mice (N=3 per treatment) received two injections of either F/T dead tumour cells “lysis (2 injection)”, γ-irradiated dead tumour cells “gamma-irr (2 injection)”, or PBS “PBS”. These were delivered via intraperitoneal injections on days −14 and −7 according to the schedule of FIG. 1. In all treatments dead cancer cells were delivered at 5.0×10⁶ cells/200 μL in PBS. On day 0 all mice were challenged with 1.0×10⁶ live B16-OVA cells in 100 μL of PBS via subcutaneous injection in the right hind flank. Tumour outgrowth was measured every second day using calipers and volume was determined according to the formula (l×w²) 2 (where w is the smaller of the two parameters). Mice were euthanized once tumour volume reached 2000 mm³.

In a further study tumour growth and survival in mice following injection with dead B16-OVA cells exposed to different methods of inducing cell death was examined. Following prophylactic vaccination delivery on day −7 and −14 of either PBS, UV exposed dead cells (“UV”), F/T dead cells (“Lysis”), or both UV and Lysis dead cells (“LyUV”), all at 5.0×10⁶ cells/200 μL in PBS, mice were challenged with 1.0×10⁶ live B16-OVA cells in 100 μL of PBS via subcutaneous injection in the right hind flank on day 0, and tumour outgrowth was measured every second day using calipers and volume was determined according to the formula (l×w²)/2 (where w is the smaller of the two parameters). Mice were euthanized once tumour volume reached 2000 mm³ (N=3). The study was repeated using the above treatments and an additional treatment of γ-irradiated tumour cells (“γ-irradiation”), N=6 for all treatments.

### Addition of Vaccine Adjuvants and Tumour Measurement

For the addition of adjuvants, B16-OVA cells were exposed to 60 Gys of γ-irradiation or UV and Lysis (“LyUV”) as described above. Following irradiation or LyUV, adjuvants were added to the cell suspension. Two studies investigated the effect of dosage of IL-27 (10 ng/mouse or 100 ng/mouse) with γ-irradiated cells (N=3 and N=at least 6 per treatment). In another study, the effect of dosage of IL-27 (10 ng/mouse or 100 ng/mouse) was investigated using LyUV cells (N=at least 5 per treatment).

In another study the effectiveness of vaccination with γ-irradiated tumour cells alone or with CpG (2 μg/mouse or 20 μg/mouse) was investigated. In another study the effectiveness of vaccination with γ-irradiated tumour cells alone or γ-irradiated tumour cells in combination with IL-27 (10 ng/mouse) and CpG-1826 (20 μg/mouse) was investigated. In another study the effectiveness of vaccination with dead tumour cells (exposed to F/T and UV (“LysUV”) alone or with CpG (20 μg/mouse), IL-27 (10 ng/mouse), or CpG (20 μg/mouse) and IL-27 (10 ng/mouse) was investigated.

In each study the vaccine was delivered following the same vaccination schedule described above and shown in FIG. 1. Tumour outgrowth was measured as described above.

### Tumour Rechallenge

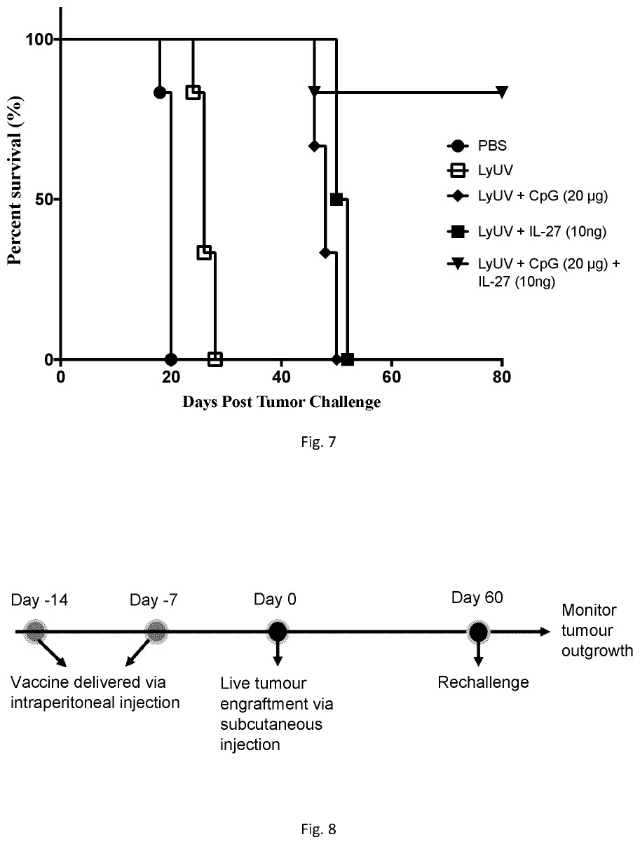

Mice that had been vaccinated as described above (FIG. 1) with γ-irradiated tumour cells and CpG (20 μg/mouse) and/or IL-27 (10 ng/mouse) or with LyUV cells and CpG (20 μg/mouse) and/or IL-27 (10 ng/mouse) and were tumour free after 60 days were subjected to a second tumour engraftment (i.e., “rechallenge”, see FIG. 8) of 1.0×10⁶ B16-OVA cells in 100 μL PBS via subcutaneous injection in the left hind flank. Tumour outgrowth was then monitored every second day until 60 days post rechallenge (100 days after initial tumour engraftment). Tumours were measured as described above.

### Investigation of Other Adjuvants

A study was conducted to investigate the effectiveness of other adjuvants in combination with IL-27. The adjuvants were endosomal TLRs which included Poly(I:C), R848, and CpG. The effects of these adjuvants in combination with IL-27 were evaluated based on dendritic cell activation, as measured by cell surface marker expression on day 7 bone marrow-derived dendritic cells (dendritic cells were identified as being CD11c+/MHC-II+). Activation was determined by an increase in at least one marker (MHC-II, CD80, CD86, MHC-I, and CD40), wherein an increase of at least one marker would confirm use of a TLR agonist in combination with IL-27 to promote an anti-tumour immune response.

### Therapeutic Application

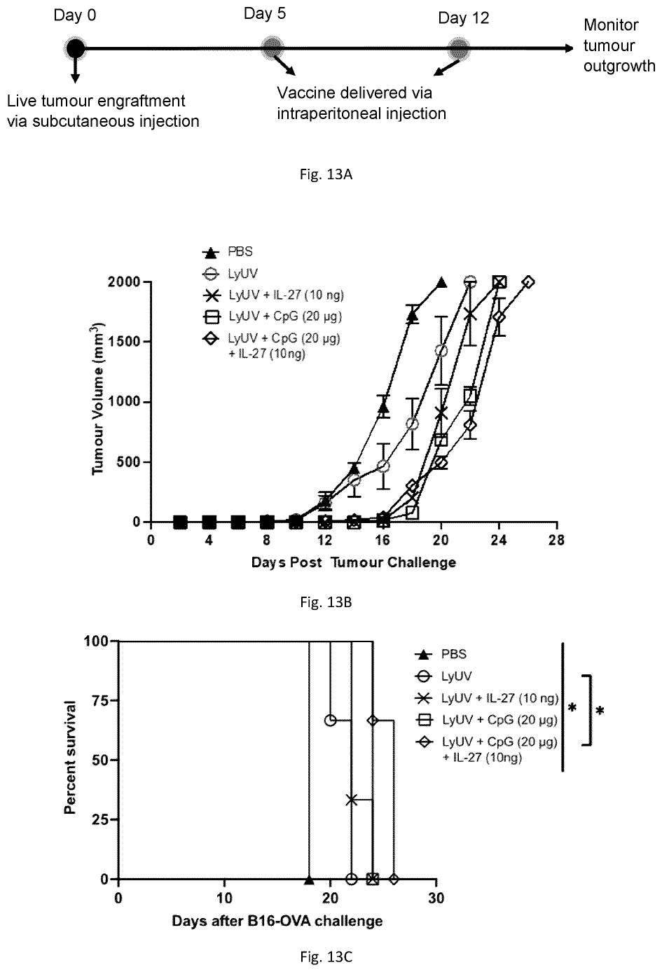

FIG. 13A is a schematic diagram of a vaccine delivery schedule implemented in mice to investigate a therapeutic application. Mice (N=3 per treatment) received PBS or a vaccine (LyUV cells, LyUV cells+IL-27 (10 ng/mouse), LyUV cells+CPG (20 ng/mouse), or LyUV cells+IL-27 (10 ng/mouse)+CPG (20 ng/mouse)) at 5 days and again at 12 days after live tumour engraftment. Tumour volume was measured as described above and mouse survival was monitored.

### Results and Discussion

### Exposure to γ-Irradiation Results in Significant Protection Against B16-OVA Tumour Challenge

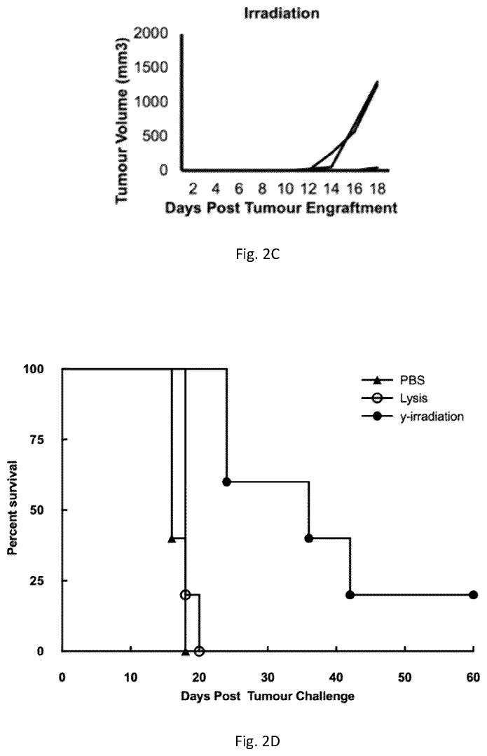

The timing and location of vaccine delivery can influence the success of a prophylactic cancer vaccine aiming to establish a robust anti-tumour immune response. For evaluating the efficacy of a prophylactic cancer vaccine, a prime/boost model of delivery as outlined in FIG. 1 was followed. After establishing the vaccination schedule, the first aim was to determine whether a prophylactic cancer vaccine consisting of B16-OVA cells exposed to 60 Gys γ-irradiation was able to provide improved protection against tumour challenge when compared to F/T. It was determined that B16-OVA cells exposed to F/T (i.e., “lysis”, FIG. 2B) failed to improve protection against tumour challenge when compared to non-vaccinated control mice (FIG. 2A). In comparison, exposure to γ-irradiation delayed the initial onset of tumour growth, with 3 mice failing to develop palpable tumours 16 days post tumour engraftment (FIG. 2C). In addition to delaying tumour growth, exposure to γ-irradiation also improved overall protection against tumour challenge, with 20% of the mice remaining tumour free for 60 days, compared to 0% of the mice in both the PBS control and lysis groups (FIG. 2D). Together these results confirm that B16-OVA cells exposed to 60 Gys of γ-irradiation is more effective at providing protection in a prophylactic cancer vaccine than that of F/T. Therefore, further experiments were conducted using tumour cells exposed to γ-irradiation as it provided a substantial improvement in protection against tumour development.

Results of the experiment to determine if the vaccine schedule could be shortened by providing only a single delivery of dead tumour cells (prepared by F/T (lysis) or γ-irradiation) are shown in the Kaplan-Meier survival curve of FIG. 2E. It can be seen that survival of mice that received a single injection or two injections of F/T dead tumour cells “lysis (1 injection)”, “lysis (2 injection)” was similar to controls that received only PBS. However, mice that received a single injection of γ-irradiated dead tumour cells “gamma-irr (1 injection)” was better than for the lysis groups, and two injections of γ-irradiated dead tumour cells “gamma-irr (2 injection)” resulted in the best survival rates.

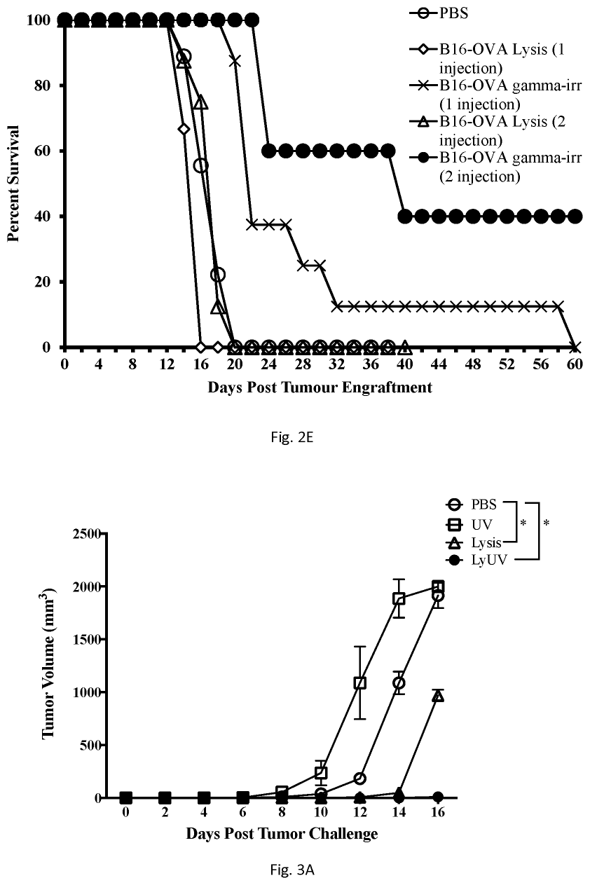

In the study investigating the method of inducing tumour cell death, tumour growth was reduced and survival was improved only in mice that received injection of dead B16-OVA cells exposed to both UV and F/T (lysis) (FIGS. 3A and 3B). This result suggests that inducing tumour cell death by either UV exposure or F/T is less effective as a vaccine when compared to γ-irradiation.

In the further study investigating the method of inducing tumour cell death, survival was improved and not significantly different in mice that received injection of dead B16-OVA cells exposed to either LyUV or γ-irradiation (FIG. 3C).

The Addition of IL-27 to a Prophylactic Cancer Vaccine Improves Vaccine Efficacy Against Initial Tumour Challenge but does not Confer Development of an Anti-Tumour Memory Response.

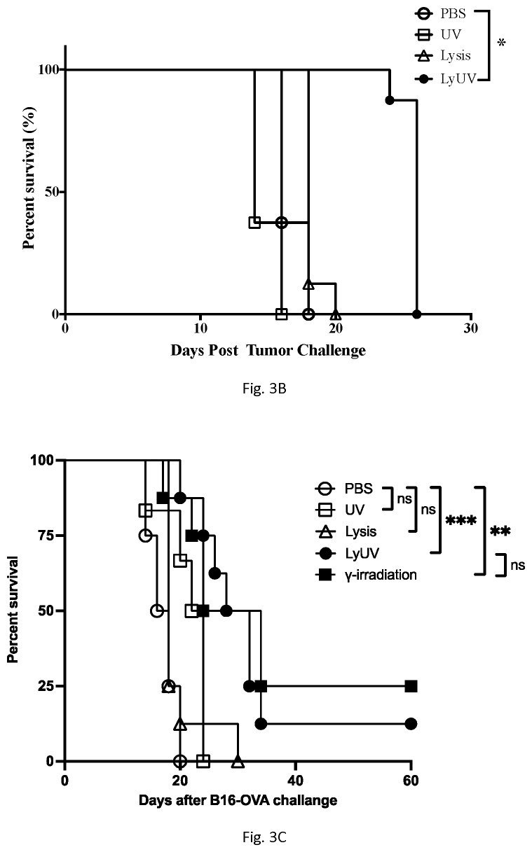

As shown in FIGS. 2C-2E, vaccination with tumour cells exposed to γ-irradiation can promote the development of an anti-tumour immune response in some mice. Further studies were conducted to determine whether the addition of IL-27 would improve the protection established by a cancer vaccine using γ-irradiated tumour cells or LyUV cells. Using the vaccination schedule of FIG. 1, IL-27 was added to the dead B16-OVA cell suspension at the time of injection (day −7 and −14) at a dosage of either 10 ng/mouse or 100 ng/mouse. It was determined that endogenous IL-27 at 10 ng/mouse was effective in providing a significant increase in protection when compared to γ-irradiation alone, with 100% of the mice remaining tumour free for 60 days after tumour engraftment (FIG. 4A). In a further study with a larger sample size, 50% of mice treated with γ-irradiated cells and IL-27 at 10 ng/mouse survived to at least 60 days (FIG. 4B). In the study using LyUV cells together with IL-27, about 55% of mice vaccinated with LyUV and IL-27 at 10 ng/mouse survived to at least 60 days (FIG. 4C). It is expected that other dosages, e.g., 25 ng/mouse, would also be effective, and that an effective dosage ranges for other subjects can be determined (e.g., scaled) according to factors such as body mass or metabolic rate as is known in the art.

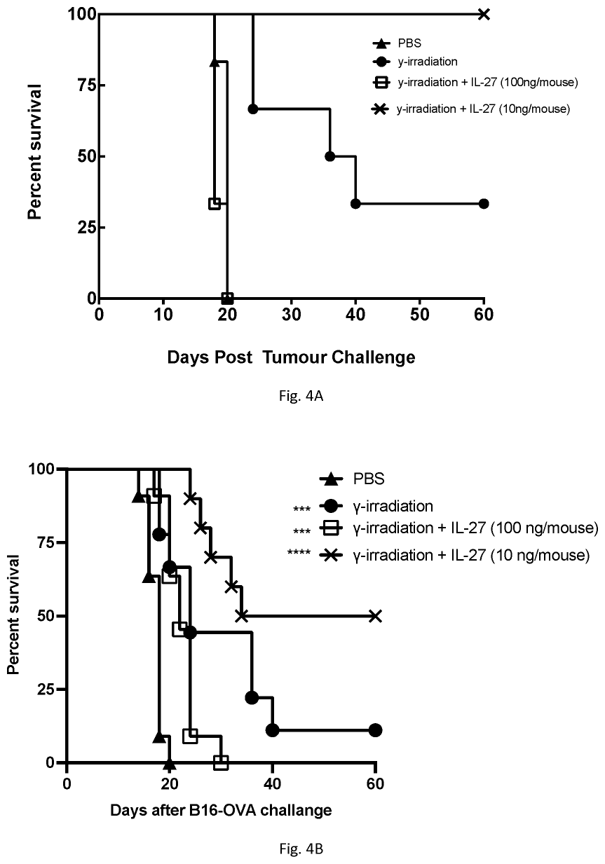

Further studies were conducted to determine whether the addition of CpG would improve the protection established by a cancer vaccine using γ-irradiated tumour cells. Using the vaccination schedule of FIG. 1, CpG was added to the dead B16-OVA cell suspension at the time of injection (day −7 and −14) at a dosage of either 2 μg/mouse or 20 μg/mouse. It was determined that CpG at 20 μg/mouse was effective in providing a significant increase in protection when compared to γ-irradiation alone, with 100% of the mice remaining tumour free for 30 days after tumour engraftment (FIG. 5A), or up to 60 days after tumour engraftment (FIG. 5B). It is expected that other dosages, e.g., 5 μg/mouse, 10 μg/mouse, would also be effective, and that effective dosages ranges for other subjects can be determined (e.g., scaled) according to factors such as body mass or metabolic rate as is known in the art.

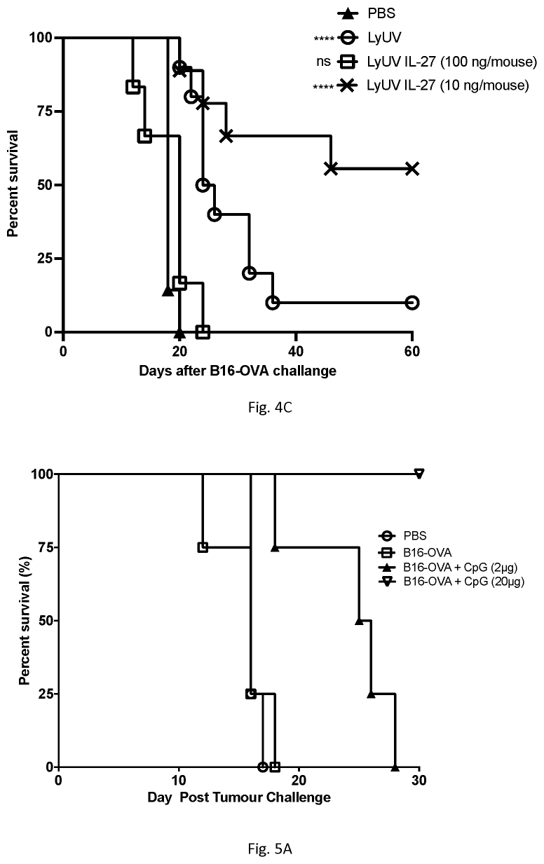

A further study was conducted to determine whether the addition of CpG and IL-27 would sustain the protection established by a cancer vaccine using γ-irradiated tumour cells. Using the vaccination schedule of FIG. 1, CpG (20 μg/mouse) and IL-27 (10 ng/mouse) were added to the dead B16-OVA cell suspension at the time of injection (day −7 and −14). It was determined that this vaccine was effective in providing a significant increase in protection when compared to γ-irradiation alone, with 100% of the mice remaining tumour free for 60 days after tumour engraftment (FIG. 6).

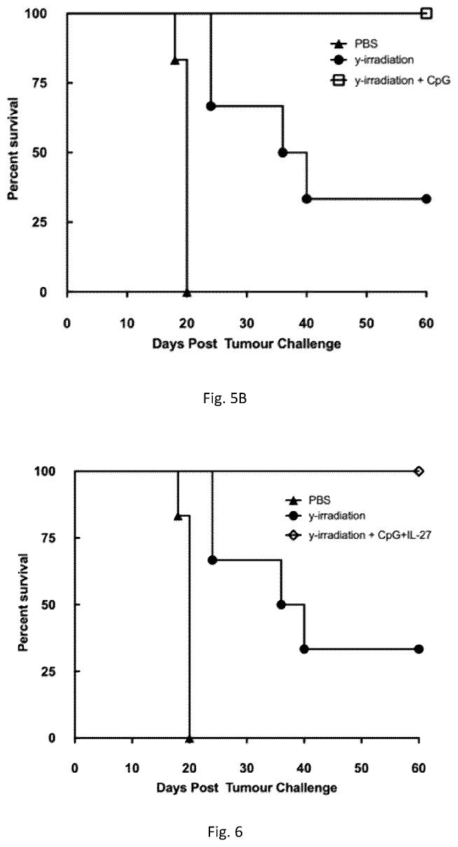

A further study was conducted to determine whether the addition of CpG and/or IL-27 would improve the protection established by a cancer vaccine using tumour cells exposed to F/T and UV (“LyUV”). Using the vaccination schedule of FIG. 1, CpG (20 μg/mouse) and/or IL-27 (10 ng/mouse) were added to the LyUV B16-OVA cell suspension at the time of injection (day 0). It was determined that none of the treatment groups resulted in 100% survival of mice by about 50 days after tumour engraftment (FIG. 7).

### The Combination of IL-27 and CpG Promote Protection Against Tumour Challenge and Produce Long Term Memory Protecting Against Tumour Rechallenge.

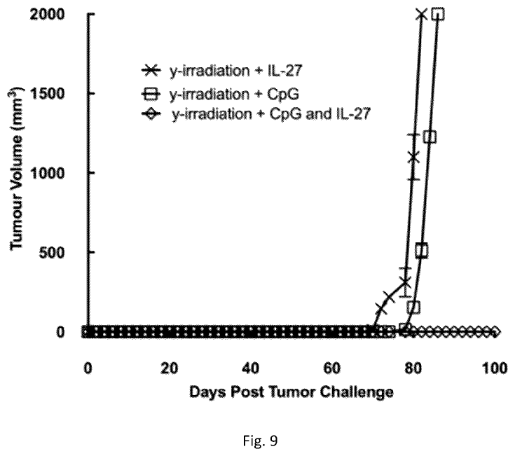

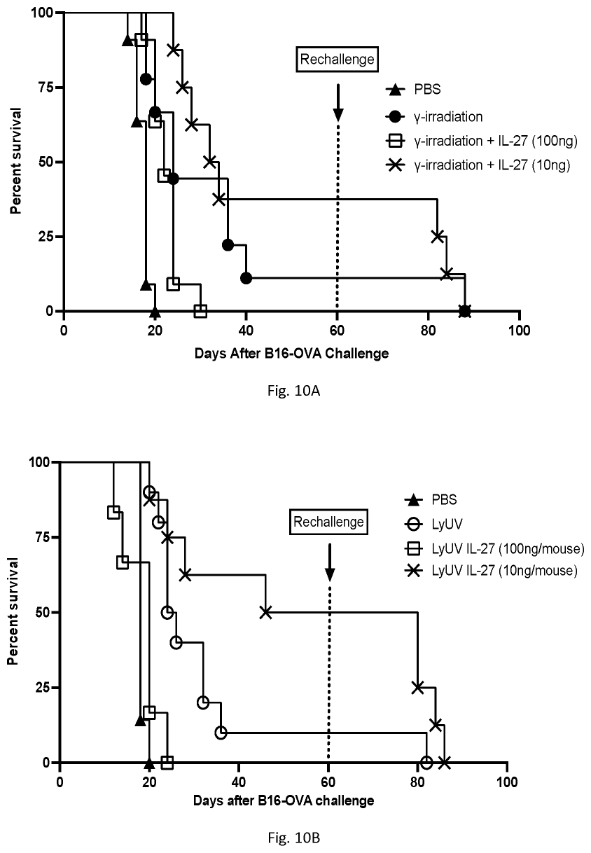

To determine whether IL-27 and/or CpG could promote the development of a memory response against tumour development when used in a vaccine with γ-irradiated tumour cells or with LyUV cells, mice that remained tumour free after the first engraftment were rechallenged with 1.0×10⁶ live B16-OVA cells on day 60 (FIG. 8). Mice of equal age (approximately 14-16 weeks) were used as controls and were challenged with live B16-OVA tumour cells at the same time. It was determined that IL-27 (10 ng/mouse) or CpG (20 g/mouse) failed to provide protection against tumour rechallenge. All mice that showed initial protection demonstrated rapid tumour growth that was equal to that of non-vaccinated control mice (FIGS. 9, 10A, and 10B). These results indicate that whereas IL-27 and CpG can be used alone to promote an initial robust anti-tumour immune response when administered with γ-irradiated tumour cells, they are not capable of developing a memory response against tumour development when used alone as evident by rechallenge experiments.

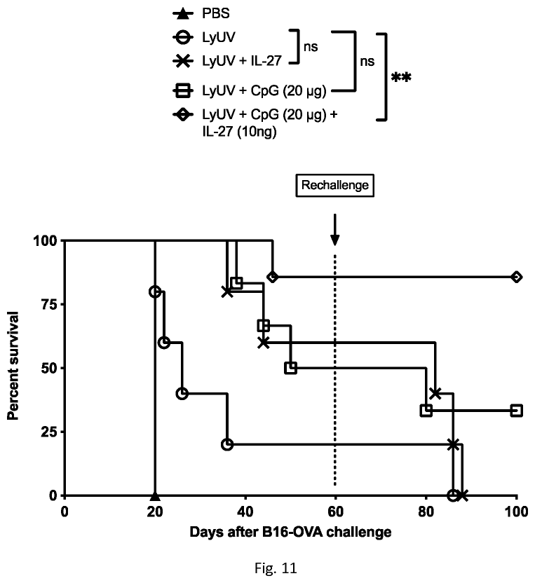

With IL-27 and CpG unable to provide long-term protection against tumour rechallenge when used independently with γ-irradiated tumour cells, the next aim was to evaluate whether the combination of these two adjuvants could improve vaccine efficacy. The combination of IL-27, CpG, and γ-irradiated tumour cells provided protection against initial tumour challenge on day 0, and upon tumour rechallenge at day 60, mice that received this combination therapy demonstrated complete protection against tumour rechallenge (FIG. 9). Also, the combination of IL-27, CpG, and LyUV cells provided protection against initial tumour challenge on day 0, and upon tumour rechallenge at day 60, mice that received this combination therapy were protected against tumour rechallenge (FIG. 11). These results indicate that the combination of CpG and IL-27 as vaccine adjuvants in a γ-irradiated tumour cell cancer vaccine or a LyUV tumour cell vaccine provides protection against tumour development and has the potential to establish an anti-tumour memory response. Furthermore, these results demonstrate the potential combination of TLR agonists and cytokines to further improve cancer vaccine efficacy.

### Other Adjuvants

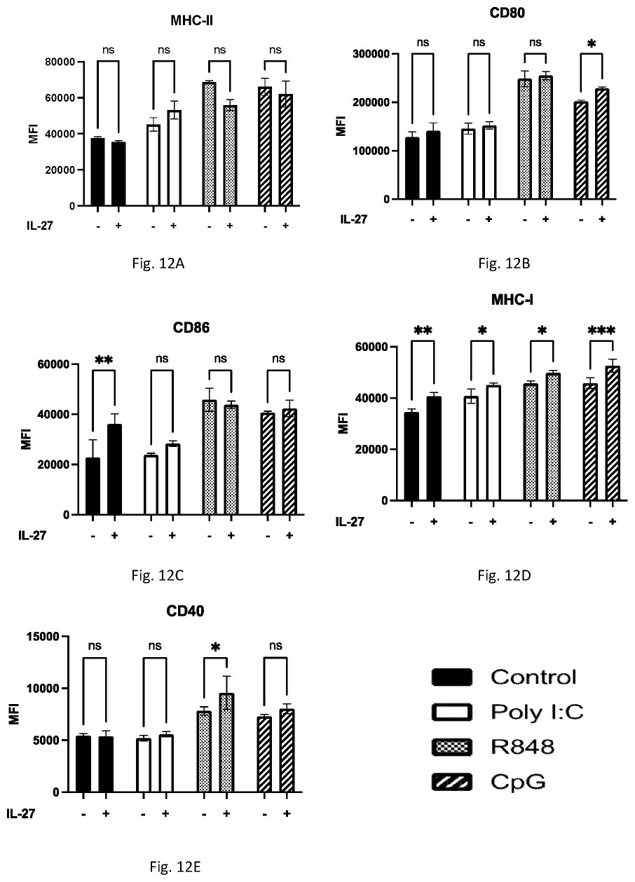

In the study investigating the effectiveness of other adjuvants in combination with IL-27, the combinations of IL-27 with Poly I:C, R848, and CpG resulted in increases of three of the cell surface markers (CD80, MHC-1, and CD40) when compared to the TLR agonist used alone (see FIGS. 12B, 12D, and 12E), indicating dendritic cell activation. This confirms use of Poly(I:C) and R848 as other TLR agonists in combination with IL-27 to promote an anti-tumour immune response.

### Discussion

Overall, results presented herein indicate that TLRs and cytokines, combined with dead tumour cells, represent a potent combination for an effective anti-tumour immune response. Embodiments based on a combination of the TLR9 agonist CpG-1826 and the cytokine IL-27 provides a combination of adjuvants for use in a prophylactic cancer vaccine based on γ-irradiated tumour cells or LyUV tumour cells. Immunization resulted in significant protection against tumour challenge, and also resulted in the development of long-term memory response. These results confirm the use of TLR agonists and cytokines as a combination immunotherapy that can be used to prevent disease recurrence, and/or promote T cell activation and memory against a particular tumour. It is expected that similar results will be obtained with other cytokine and TLR agonist combinations and in other tumour models, and in therapeutic models to reduce disease progression.

Based on the results, an exemplary therapeutic treatment regime was investigated based on the schedule shown in FIG. 13A. Mice (N=3 per treatment) received PBS or a vaccine (LyUV cells, LyUV cells+IL-27 (10 ng/mouse), LyUV cells+CPG (20 ng/mouse), or LyUV cells+IL-27 (10 ng/mouse)+CPG (20 ng/mouse)) at 5 days and again at 12 days after live tumour engraftment. Rate of tumour growth was slowed and survival was increased by the LyUV vaccines that included an adjuvant. Mice that were vaccinated with LyUV cells+IL-27 (10 ng/mouse)+CPG (20 ng/mouse) had the slowest tumour growth and survived the longest. It is expected that a treatment regime based on embodiments as described herein may be adapted for other mammals, including humans, and may provide a therapeutic benefit by preventing, inhibiting, or slowing tumour development and improving survival in cancer patients.

For example, in one embodiment a treatment regime may use tumour cells removed from a patient (i.e., autologous whole tumour cell vaccine), such as, for example, solid and hematological margins in which tumour cells can be isolated (e.g., colon, prostate, pancreatic, breast, melanoma, lekemia, myeloma, lymphoma, etc.). The isolated tumour cells may be killed, e.g., by subjecting them to LyUV or γ-irradiation, and the dead tumour cells used in a vaccine together with adjuvants including at least one Toll-like receptor (TLR) agonist, at least one cytokine, and a pharmaceutically acceptable vehicle, which is then administered back to the same patient to prevent, inhibit, or slow tumour development and improve survival in the patient.

In other embodiments the cancer cells are not isolated from the patient being treated. Instead, the dead cancer cells used in the vaccine are of a type that may be similar to the type of cancer in the patient (i.e., allogeneic). Here, “similar” means that the dead tumour cells provided to the patient in the vaccine have at least one similar or same TAA as the tumour cells in the patient. These may be one or more TAAs that may be conserved among different tumour types, examples of which include, but are not limited to, MAGE, NY-ESO-1, HER2, mesothelin, TPD52, and MUC1. Dead cancer cells may also be obtained from a cancer cell line that provides at least one selected TAA. As a non-limiting example, one such tumour cell line is B16-OVA used in embodiments described herein.

In other embodiments, a cancer vaccine as described herein may be administered prophylactically or in a target-directed regime in subjects who are susceptible or predisposed to developing cancer or to prevent cancer recurrence in patients after treatment. For example, aberrant expression of MUC1 occurs in cancer cells, including those of oesophageal cancer, gastric cancer, breast cancer, ovarian cancer, bladder cancer, colon cancer, and other tumors, and is especially prominent in breast cancer cells. Such patients may be treated with a vaccine that includes dead cells expressing MUC1 and with adjuvants including at least one Toll-like receptor (TLR) agonist, at least one cytokine, and a pharmaceutically acceptable vehicle. Furthermore, subjects predisposed to developing cancer based on a mutation in a specific gene (e.g., BRCA1) may be treated prophylactically by administering a vaccine including dead cancer cells that express a specific TAA that is commonly found with the cancer type arising from the identified mutation with adjuvants including at least one Toll-like receptor (TLR) agonist, at least one cytokine, and a pharmaceutically acceptable vehicle.

### INCORPORATION BY REFERENCE

The contents of all cited publications are incorporated herein by reference in their entirety.

### EQUIVALENTS

It will be appreciated that modifications may be made to the embodiments described herein without departing from the scope of the invention. Accordingly, the invention should not be limited by the specific embodiments set forth, but should be given the broadest interpretation consistent with the teachings of the description as a whole.

### REFERENCES

1. Pedersen, S. R., et al., Comparison of vaccine-induced effector CD8 T cell responses directed against self- and non-self-tumor antigens: implications for cancer immunotherapy. J Immunol, 2013. 191(7): p. 3955-67.
2. Peng, M., et al., Neoantigen vaccine: an emerging tumor immunotherapy. Mol Cancer, 2019. 18(1): p. 128.
3. Schumacher, T. N. and R. D. Schreiber, Neoantigens in cancer immunotherapy. Science, 2015. 348(6230): p. 69-74.
4. Buonaguro, L., et al., Translating tumor antigens into cancer vaccines. Clin Vaccine Immunol, 2011. 18(1): p. 23-34.
5. Chiang, C. L., G. Coukos, and L. E. Kandalaft, Whole tumor antigen vaccines: Where are we? Vaccines (Basel), 2015. 3(2): p. 344-72.
6. Capitini, C. M., T. J. Fry, and C. L. Mackall, Cytokines as adjuvants for vaccine and cellular therapies for cancer. Am J Immunol, 2009. 5(3): p. 65-83.
7. Temizoz, B., E. Kuroda, and K. J. Ishii, Vaccine adjuvants as potential cancer immunotherapeutics. Int Immunol, 2016. 28(7): p. 329-38.
8. Mohan, T., et al., Applications of chemokines as adjuvants for vaccine immunotherapy. Immunobiology, 2018. 223(6-7): p. 477-485.
9. van der Zanden, S. Y., et al., Opportunities for small molecules in cancer immunotherapy. Trends Immunol, 2020. 41(6): p. 493-511.
10. Adams, S., Toll-like receptor agonists in cancer therapy. Immunotherapy, 2009. 1(6): p. 949-64.
11. Kaczanowska, S., A. M. Joseph, and E. Davila, TLR agonists: our best frenemy in cancer immunotherapy. J Leukoc Biol, 2013. 93(6): p. 847-63.
12. Shirota, H., D. Tross, and D. M. Klinman, CpG oligonucleotides as cancer vaccine adjuvants. Vaccines (Basel), 2015. 3(2): p. 390-407.
13. Portielje, J. E., et al., IL-12: a promising adjuvant for cancer vaccination. Cancer Immunol Immunother, 2003. 52(3): p. 133-44.
14. Chang, D. Z., et al., Granulocyte-macrophage colony stimulating factor: an adjuvant for cancer vaccines. Hematology, 2004. 9(3): p. 207-15.
15. Bowen, W. S., et al., Current challenges for cancer vaccine adjuvant development. Expert Rev Vaccines, 2018. 17(3): p. 207-215.
16. Miguel, A., et al., Comparative antitumor effect among GM-CSF, IL-12 and GM-CSF+IL-12 genetically modified tumor cell vaccines. Cancer Gene Ther, 2013. 20(10): p. 576-81.
17. Lasek, W., R. Zagozdzon, and M. Jakobisiak, Interleukin 12: still a promising candidate for tumor immunotherapy? Cancer Immunol Immunother, 2014. 63(5): p. 419-35.
18. Kourko, O., et al., IL-27, IL-30, and IL-35: A cytokine triumvirate in cancer. Front Oncol, 2019. 9: p. 969.
19. Overwijk, W. W. and Restifo, N. P., B16 as a mouse model for human melanoma. Curr. Protoc. Immunol. 2001 May; Unit 20.1, doi: 10.1002/0471142735.im2001s39

1. A composition, comprising:
at least one tumour associated antigen (TAA);
at least one Toll-like receptor (TLR) agonist;
at least one cytokine; and
a pharmaceutically acceptable vehicle.

2. The composition of claim 1, wherein the composition is a cancer vaccine.

3. The composition of claim 1, wherein the at least one TAA comprises dead tumour cells.

4. The composition of claim 1, wherein the at least one TLR agonist comprises at least one TLR9 agonist.

5. The composition of claim 4, wherein the at least one TLR9 agonist comprises a CpG oligodeoxynucleotide (CpG-ODN).

6. The composition of claim 5, wherein the at least one TLR9 agonist comprises CpG-1826.

7. The composition of claim 1, wherein the at least one cytokine comprises an interleukin (IL).

8. The composition of claim 7, wherein the at least one cytokine comprises IL-27.

9. The composition of claim 2, wherein the cancer vaccine prevents, inhibits, or slows melanoma development.

10. The composition of claim 4, wherein the at least one TLR9 agonist comprises CpG-1826 and the at least one cytokine comprises IL-27.

11. The composition of claim 3, wherein the dead tumour cells comprise γ-irradiated tumour cells.

12. The composition of claim 3, wherein the dead tumour cells comprise lysis and UV treated tumour cells.

13. The composition of claim 2, wherein the dead tumour cells comprise γ-irradiated B16 tumour cells, and the cancer vaccine prevents, inhibits, or slows melanoma development.

14. A method for preventing, inhibiting, or slowing tumour development, comprising:
providing a composition comprising at least one tumour associated antigen (TAA), at least one Toll-like receptor (TLR) agonist, at least one cytokine, and a pharmaceutically acceptable vehicle;
administering an effective amount of the composition to a subject;
wherein the composition prevents, inhibits, or slows the tumour development in the subject.

15. The method of claim 14, wherein the at least one TAA comprises dead tumour cells.

16. The method of claim 14, wherein the at least one TLR agonist comprises at least one TLR9 agonist.

17. The method of claim 16, wherein the at least one TLR9 agonist comprises a CpG oligodeoxynucleotide (CpG-ODN).

18. The method of claim 17, wherein the at least one TLR9 agonist comprises CpG-1826.

19. The method of claim 14, wherein the at least one cytokine comprises an interleukin (IL).

20. The method of claim 19, wherein the interleukin comprises IL-27.

21. The method of claim 14, wherein the at least one TLR agonist comprises CpG-1826 and the at least one cytokine comprises IL-27.

22. The method of claim 15, wherein the dead tumour cells comprise γ-irradiated tumour cells.

23. The method of claim 15, wherein the dead tumour cells comprise lysis and UV treated tumour cells.

24. The method of claim 15, wherein the dead tumour cells comprise γ-irradiated B16 tumour cells and the composition prevents, inhibits, or slows melanoma development in the subject.

25. The method of claim 24, wherein the composition provides a long-term T cell activation and memory against B16 tumour development in the subject.
